SEQUENCE LISTING (1 sequence)

SEQ ID NO: 1 - protein, 9 aa, Artificial Sequence
  Synthetic
YLSGANLNL

(19) United States
(12) Patent Application Publication
(10) Pub. No.: US 2017/0306042 A1
(43) Pub. Date: Oct. 26, 2017

(54) IMMUNOGENIC MODULATION BY ENDOCRINE DEPRIVATION THERAPY IMPROVES SENSITIVITY OF TUMOR CELLS TO IMMUNE MEDIATED LYSIS

(71) Applicant: The United States of America, as represented by the Secretary, Department of Health and Human Serv, Bethesda, MD (US)

(72) Inventors: Jeffrey Schlom, Potomac, MD (US); James W. Hodge, Kensington, MD (US)

(73) Assignee: The United States of America, as represented by the Secretary, Department of Health and Human Serv, Bethesda, MD (US)

(21) Appl. No.: 15/507,316

(22) PCT Filed: Aug. 28, 2015
(86) PCT No.: PCT/US2015/047538

§ 371 Date: Feb. 28, 2017

Related U.S. Application Data
(60) Provisional application No. 62/043,880, filed on Aug. 29, 2014.

(51) Int. Cl. C07K 16/30; A61K 39/395; A61K 31/517; A61K 45/06; C07K 16/28

(52) U.S. Cl. CPC C07K 16/30; A61K 45/06; C07K 16/28; A61K 31/517; A61K 39/39558; C07K 2317/76

(57) ABSTRACT

The invention is directed to methods of reducing growth of prostate cancer cells and breast cancer cells, which comprises treating such cancer cells with a combination of androgen or endocrine deprivation therapy (e.g., enzalutamide, abiraterone, and tamoxifen) and immunotherapy.

### CROSS-REFERENCE TO RELATED APPLICATIONS

This patent application claims the benefit of U.S. Provisional Patent Application No. 62/043,880 filed Aug. 29, 2014, which is incorporated by reference.

### SEQUENCE LISTING

Incorporated by reference in its entirety herein is a nucleotide/amino acid sequence listing submitted concurrently herewith.

### BACKGROUND OF THE INVENTION

Endocrine deprivation therapy is the standard of care for prostate cancer (Huggins, Cancer Res., 27(11):1925-1930 (1967); and Huggins, C., Arch. Surg. (Chicago), 43: 209-223 (1941)) and breast cancer. The agents enzalutamide and abiraterone have been approved by the U.S Food and Drug Administration (FDA) for the treatment of castration resistant prostate cancer (CRPC), and target androgen receptor (AR) signaling or testosterone production. Enzalutamide is an androgen receptor (AR) antagonist that blocks androgens from binding to the AR and prevents nuclear translocation and coactivator recruitment of the ligand-receptor complex. The utility of enzalutarnide has been demonstrated in clinical trials (Tran et al., Science, 324(5928): 787-790 (2009); Scher et al., Lancet, 375(9724): 1437-1446 (2010); and Scher et al., J. Clin. Oncol., 30(suppl5): abstr LBA1 (2012)), including the AFFIRM trial where it mediated a 4.8-month advantage in overall survival compared to placebo (Scher et al., J. Clin. Oncol., 30(suppl5): abstr LBA1 (2012)). Abiraterone is a potent inhibitor of CYP17A1, a rate-limiting enzyme in androgen biosynthesis. Inhibition of CYP17A1 subsequently blocks the production of androgen in all endocrine organs, including the testes, adrenal glands, and in the prostate tumor itself (Harris et al., Nature Clinical Practice Urology, 6(2):76-85 (2009)). In a phase III study in patients with CRPC previously treated with docetaxel, abiraterone was shown to improve overall survival by 3.9 months compared to placebo (de Bono et al., New Eng. J. Med., 364(21): 1995-2005 (2011)).

Tamoxifen blocks estrogen and has been used as the first line hormonal therapy for breast cancer for over 30 years. Enzalutamide and Abiraterone also are being evaluated in breast cancer.

Despite the advances in endocrine deprivation therapy for prostate cancer and breast cancer, patients often develop resistance to these therapies. For example, in reported clinical trials, more than 30% of prostate cancer patients did not respond to enzalutamide and continued to have rising PSA levels (Scher et al., Lancet, 375(9724): 1437-1446 (2010); and Gameiro et al., Cancer Immunology, Immunotherapy, 60(9):1227-1242 (2011)). Moreover, nearly all patients who initially respond to ADT will ultimately develop ADT resistance (Karantanos et al., Oncogene, 32(49): 5501-5511(2013)).

There is a need for improved methods for inhibiting growth and progression of castration-resistant prostate cancer and breast cancer. The invention provides such methods.

### BRIEF SUMMARY OF THE INVENTION

The invention provides a method of reducing prostate cancer cell growth, which method comprises treating prostate cancer cells with a combination of androgen deprivation therapy and immunotherapy, whereupon growth of the prostate cancer cells is reduced.

The invention also provides a method of reducing prostate cancer cell growth, which method comprises treating prostate cancer cells with a combination of abiraterone and immunotherapy, whereupon growth of the prostate cancer cells is reduced.

The invention provides a method of reducing breast cancer cell growth, which method comprises treating breast cancer cells with a combination of endocrine deprivation therapy and immunotherapy, whereupon growth of the breast cancer cells is reduced.

### BRIEF DESCRIPTION OF THE SEVERAL VIEWS OF THE DRAWING(S)

FIGS. 1A-1H are graphs depicting experimental results which illustrate that ADT inhibited the growth of AR+ prostate tumor cells and improved their sensitivity to T-cell-mediated killing. The human prostate tumor cell lines LNCaP (AR+; HLA-A2) (A) and PC-3 (AR−, HLA-A24) (C) were treated with vehicle (DMSO; open symbols) or 10 μM enzalutamide (closed symbols). Cell proliferation was determined at the indicated time points. After 48 hours of either vehicle or enzalutamide treatment, LNCaP (B) and PC-3 (D) cells were used as targets in a CTL lysis assay using MUC1-specific CD8+ T-cells as effector cells at an E:T ratio of 30:1. (B) Inset, LNCaP cells treated with vehicle or enzalutamide were used as CTL targets in the presence of an anti-HLA-A2 blocking antibody. LNCaP and PC-3 cells were treated with vehicle (open circles) or 10 μM abiraterone (closed circles). (E-H) The effect of abiraterone on LNCaP (E-F) and PC-3 (G-H) cell proliferation and CTL sensitivity to MUC1-specific CD8+ T-cells as effector cells was determined. Viability of the cells tested was assessed at 72 hours after treatment by trypan blue exclusion (insets). Results are presented as mean±S.E.M. from 3-6 replicate wells. Asterisks denote statistical significance relative to controls (*P<0.05, **P<0.01). These experiments were repeated 3-5 times with similar results.

FIGS. 2A-2D are graphs depicting experimental results which illustrate that increased CTL sensitivity induced by enzalutamide was dependent on AR expression. Human prostate tumor cell lines LNCaP expressing control-shRNA (AR+, HLA-A2) (A) and LNCaP AR-shRNA (AR−, HLA-A2) (B) were treated with vehicle (DMSO; open symbols) or 10 μM enzalutamide (closed symbols). Cell proliferation was determined at the indicated time points. AR expression levels were confirmed by RT-PCR (inset). After 48 hours of either vehicle or enzalutamide treatment, LNCaP control-shRNA (C) and LNCaP AR-shRNA (D) cells were used as targets in a CTL lysis assay using CEA-specific CD8+ T-cells as effector cells at an E:T ratio of 30:1 (bottom panels). Results are presented as mean±S.E.M. from 3-6 replicate wells. Asterisks denote statistical significance relative to controls (*P<0.05, **P<0.01, ***P<0.001, NS: not significant). These experiments were repeated 3-5 times with similar results.

FIGS. 3A and 3B are graphs depicting experimental results which illustrate that enzalutamide mediated reduced PSA levels while improving prostate tumor-cell sensitivity to PSA-specific CD8+ T-cell killing. (A) Expression of PSA was analyzed by RT-PCR in LNCaP (AR+, HLA-A2) cells treated with either vehicle (DMSO) or 10 μM enzalutamide. (B) After 48 hours of either vehicle or enzalutamide treatment, cells were used as targets in a CTL lysis assay using PSA-specific CD8+ CTLs as effector cells at an E:T ratio of 30:1. Results are presented as mean±S.E.M. from 3-6 replicate wells. Asterisks denote statistical significance relative to controls (**P<0.01, ****P<0.0001). This experiment was repeated 3-5 times with similar results.

FIGS. 4A and 4B are images depicting mouse prostate xenografts illustrating that enzalutamide reduced expression of NAIP in vivo. Nude mice bearing LNCaP (AR+, HLA-A2) (A) or PC-3 (AR−, HLA-A24) (B) prostate xenografts were left untreated or treated with 10 mg/day of enzalutamide. After 7 days of treatment, mice were sacrificed, tumors were surgically removed, and expression of NAIP was detected by immunohistochemistry (magnification 20×) and quantified by positive pixel quantification analysis. Staining intensities are depicted in pie charts. Numbers indicate percentage of cells with negative, weak positive, or strong positive expression of NAIP. Insets are from isotype controls. This experiment was performed twice independently and similar results were obtained.

FIGS. 5A and 5B are graphs depicting experimental results which illustrate that silencing NAIP expression increased AR+ and AR− prostate tumor cells' sensitivity to CD8+ T-cell killing. LNCaP (AR+, HLA-A2) (A) and PC-3 (AR−, HLA-A24) (B) prostate cancer cells were treated with vehicle (DMSO) or 10 μM enzalutamide (left panels) or treated with control siRNA, NAIP siRNA, or DAPK1 siRNA (right panels) for 48 hours and used as targets in a CTL lysis assay using CEA-specific or MUC1-specific CD8+ T-cells, respectively, as effector cells at an E:T ratio of 30:1. NAIP expression after tumor cells were treated with control or NAIP siRNA was detected by western blot (insets). Results are presented as mean±S.E.M. from 3-6 replicate wells. Asterisks denote statistical significance relative to controls (***P<0.001, ****P<0.0001). This experiment was repeated 3-5 times with similar results.

FIGS. 6A-6D are graphs depicting experimental results which illustrate that enzalutamide improved the sensitivity of prostate tumor cells that overexpressed AR to T-cell-mediated killing. Overexpression of AR in the cell line LNCaP/AR(cs) was determined by RT-PCR (panel A inset). LNCaP (A) and LNCaP/AR(cs) (C) cells were treated with vehicle (DMSO; open symbols), 10 μM (closed symbols), or 30 μM enzalutamide (grey symbols). Cell proliferation was determined at the indicated time points. After 48 hours of either vehicle or enzalutamide treatment, LNCaP (B) and LNCaP/AR(cs) (D) cells were used as targets in a CTL lysis assay using CEA- or PSA-specific CD8+ T-cells as effector cells at an E:T ratio of 30:1. Asterisks denote statistical significance relative to controls (**P<0.01, ***P<0.001, ****P<0.0001).

FIGS. 7A-7D are graphs depicting experimental results which illustrate that enzalutamide inhibits the growth of androgen receptor positive breast cancer cells. (A) AR expression by ZR75-1, BT549 and MDA MB 231 cells as determined by quantitative RT-PCR and Western Blot. Breast cancer cells (B) ZR75-1, (C) BT549 and (D) MDA MB 231 were exposed to enzalutamide (closed squares) or vehicle (DMSO, open squares) for 1, 2, and 3 days then assayed for growth and viability. Error bars indicate mean±S.E.M. for quadruplicate measurements. Statistical analyses were done by Student's t-test, *=P<0.05 vs. vehicle control. Data are representative of 3 independent experiments.

FIGS. 8A-8D are graphs depicting experimental results which illustrate that enzalutamide increases the sensitivity of breast cancer cells to T cell and TRAIL mediated killing regardless of androgen receptor expression. (A) ZR75-1 (AR+, ER+), (B) BT549 (AR+, TNBC) and (C) MDA MB 231 (AR−, TNBC) cells were treated with either enzalutamide or vehicle then used as targets in a CTL assay using CEA-specific CD8+T cells as effector cells at an E:T ratio of 30:1. (D) MDA MB 231 (AR−, TNBC) cells, treated with either enzalutamide or vehicle, were used as targets in a TRAIL-mediated lysis assay. Error bars indicate mean±S.E.M. for quadruplicate measurements. Statistical analyses were done by Student's t-test, *=P<0.05 vs. vehicle control. Data are representative of 2-4 independent experiments.

FIGS. 9A and 9B are graphs depicting experimental results which illustrate that abiratirone increases the sensitivity of breast cancer cells to T cell-mediated lysis regardless of androgen receptor expression. (A) ZR75-1 (AR+, ER+) and (B) MDA MB 231 (AR−, TNBC) cells were treated with either abiraterone or vehicle then used as targets in a CTL assay using CEA-specific CD8+ T cells as effector cells at an E:T ratio of 30:1. Error bars indicate mean±S.E.M. for quadruplicate measurements. Statistical analyses were done by Student's t-test, *=P<0.01 vs. vehicle control. Data are representative of 2 independent experiments.

FIGS. 10A and 10B are graphs depicting experimental results which illustrate that: Enzalutamide significantly downregulates the expression of osteoprotegerin (OPG) in MDA-MB-231 (AR−, TNBC) cells. (A) MDA MB 231 cells were treated with enzalutamide or vehicle for 24 hours. Changes of >2-fold in apoptotic gene expression relative to vehicle control were determined by quantitative RT-PCR. (B) MDA MB 231 cells were treated with 10 μM enzalutamide or vehicle for 48 hours. Levels of OPG in the supernatant of MDA MB 231 cells as determined by ELISA. Data are representative of 2 independent experiments.

FIGS. 11A-11C are graphs depicting experimental results which illustrate that knocking down OPG expression recapitulates the increased sensitivity of MDA-MB-231 cells to T cell and TRAIL mediated killing. MDA-MB-231 (AR−, TNBC) cells were transfected with control or OPG siRNA. (A) Amount of OPG in the supernatant of MDA-MB-231 cells 48 hours after siRNA transfection. Sensitivity of MDA-MB-231 cells to (A) CEA-specific CD8+ T cell-mediated lysis or (B) TRAIL-mediated lysis cells 48 hours after siRNA transfection. Error bars indicate mean±S.E.M. for quadruplicate measurements. Statistical analyses were done by Student's t-test, *=P<0.05 vs. vehicle control. Data are representative of two independent experiments.

FIGS. 12A-12D are graphs depicting experimental results which illustrate the viability of breast cancer cells following treatment with 1 μM tamoxifen or vehicle. The fold increase in cell number is illustrated on the y-axis and hours post treatment is illustrated on the x-axis for ER positive cells (A) ZR75-1 or (C) MCF-7 and ER negative cells (B) HCC1806 and (D) BT549.

FIGS. 13A-13D are graphs depicting experimental results which illustrate that tamoxifen treatment influences the CTL sensitivity of breast cancer cells regardless of ER expression. (A) ZR75-1, (B) MCF-7, (C) BT549, and (D) HCC1806 cells were treated with either tamoxifen or vehicle then used as targets in a CTL assay using CEA-specific or MUC-1 specific CTLs as effector cells at an E:T ratio of 30:1.

### DETAILED DESCRIPTION OF THE INVENTION

The invention is predicated, at least in part, on the discovery that certain endocrine deprivation therapies induce immunogenic modulation in prostate and breast cancer cells, which sensitizes these cells to T-cell-mediated lysis.

Immunogenic modulation describes a cascade of phenotypic and molecular events that occur when conventional therapies alter the phenotype of tumor cells, rendering them more susceptible to immune-mediated attack (Kwilas et al., Frontiers in Oncology, 2:104 (2012)). The molecular mechanisms of immunogenic modulation include: (a) changes in the surface phenotype of cancer cells, including exposure of calreticulin on the outer leaflet of the plasma membrane, (b) down-regulation of antiapoptotic and/or prosurvival genes, and (c) modulation of components of the antigen-processing machinery (APM) (Gameiro et al., Oncotarget, 5(2): 403-416 (2014); Hodge et al., Seminars in Oncology, 39(3):323-339 (2012); Gameiro et al., Cancer Immunology, Immunotherapy, 60(9): 1227-1242 (2011); Gameiro et al., PloS One; 8(7):e70417 (2013); Hodge et al., International J. Cancer, 133(3): 624-636 (2013); Reits et al., J. Exp. Med., 203(5):1259-1271 (2006); and Gelbard et al., Clinical Cancer Res., 12(6):1897-1905 (2006))

The invention provides a method of reducing growth of prostate cancer cells. The method comprises treating prostate cancer cells with a combination of androgen deprivation therapy and immunotherapy, whereupon growth of the prostate cancer cells is reduced. The term “prostate cancer,” which is also synonymous with the term “prostate carcinoma,” refers to cancer that fauns in tissues of the prostate. “Prostate cancer cells” refer to cells obtained or derived from a prostate cancer. In another embodiment, the inventive method can be used to inhibit growth of hyperplastic, but not malignant, prostate cells, such as, for example, high grade prostatic intraepithelial neoplasia (HGPIN) or benign prostatic hyperplasia (BPH), which is also referred to in the art as benign enlargement of the prostate (BEP), adenofibromyomatous hyperplasia, and benign prostatic hypertrophy.

The prostate cancer cells can be of any grade or stage, as determined by histopathology and the Gleason score, and/or in accordance with the guidelines described in, e.g., Edge et al. (eds.), American Joint Committee on Cancer (AJCC) Staging Manual, 7^{th} Edition (2010), or the SEER Program Coding and Staging Manual, NIH Publication Number13-5581, U.S. Department of Health and Human Services National Cancer Institute (2013).

In one embodiment, the prostate cancer cells can have been subjected to one or more prostate cancer therapies (e.g., surgery, chemotherapy, androgen deprivation therapy, and/or radiation) prior to the inventive method. In this respect, most hormone-dependent prostate cancers become refractory to androgen deprivation therapy after one to three years and resume growth despite androgen deprivation therapy. Such cancers are known as castration resistant prostate cancer (CRPC). The prostate cancer cells can be metastatic castration resistant prostate cancer cells, which are resistant to treatment with androgen deprivation therapy alone. In another embodiment, the prostate cancer cells have become resistant to other standard treatment regimens. For example, the prostate cancer cells can be resistant to chemotherapy and/or radiation therapy.

In one embodiment, the prostate cancer cells express an androgen receptor (AR). The androgen receptor (AR), also known as NR3C4 (nuclear receptor subfamily 3, group C, member 4), is a nuclear receptor that is activated by binding of either of the androgenic hormones testosterone or dihydrotestosterone in the cytoplasm, and is translocated into the nucleus where it functions as a DNA-binding transcription factor (Roy et al., Vitamins & Hormones, 55: 309-352 (1999)). AR signaling plays a critical role in the development, function, and homeostasis of the prostate. Prostate cancer initiation and progression also is dependent on AR (Lonergan P E, Tindall D J., J. Carcinog., 10: 20 (2011)). AR expression is maintained throughout prostate cancer progression, and the majority of androgen-independent or hormone refractory prostate cancers express AR. Mutation of AR may contribute to the progression of prostate cancer and the failure of endocrine therapy by allowing AR transcriptional activation in response to antiandrogens or other endogenous hormones (Heinlein and Chang, Endocr. Rev., 25(2): 276-308 (2004)). AR also is widely expressed in breast cancers and has been proposed as a therapeutic target in estrogen-receptor (ER) negative breast cancers that express AR (Cochrane et al., Breast Cancer Res., 16: R7 (2014)).

The term “androgen deprivation therapy (ADT),” as used herein, refers to a treatment for cancer in which the level of androgen hormones, such as testosterone, in a patient are reduced, typically by pharmaceutical or surgical methods (see, e.g., Perlmutter and Lepor, Rev. Urol., 9 (Suppl 1): S3-8 (2007)). Surgical approaches to ADT include surgical castration. Pharmaceutical approaches to ADT include androgen inhibitors (antiandrogens) and chemical castration. ADT also is referred to in the art as androgen suppression therapy. Androgen inhibitors used in prostate cancer can be steroidal or non-steroidal (also referred to as “pure” antiandrogens). Steroidal androgen inhibitors include, for example, e.g., megestrol (MEGACE™), cyproterone acetate, abiraterone, and abiraterone acetate (ZYTIGA™). Non-steroidal androgen inhibitors include, for example, bicalutamide (CASODEX™), flutamide (EULEXIN™), nilutamide (ANANDRON™and NILANDRON™), and enzalutamide (XTANDI™).

In one embodiment, the androgen deprivation therapy is enzalutamide. Enzalutamide (marketed as XTANDI™ by Medivation and Astellas and formally known as MDV3100) is an oral non-steroidal small molecule androgen receptor inhibitor that prolongs survival in men with metastatic castration resistant prostate cancer in whom the disease has progressed after chemotherapy. Preclinical studies also suggest that enzalutamide also inhibits breast cancer cell growth (see, e.g., Cochrane et al., Cancer Research, 72(24 Suppl): Abstract nr P2-14-02 (2012)).

Immunogenic modulation by enzalutamide has been described in murine prostate carcinomas (see, e.g., Ardiani et al., Clinical Cancer Res., 19(22): 6205-6218 (2013)), where enzalutamide up-regulated MHC-I and Fas on the surface of tumor cells, thus improving the cells' sensitivity to T-cell killing. In these studies, treatment with enzalutamide did not alter the number or function of T-cells. Enzalutamide-mediated immunogenic modulation increased the efficacy of a therapeutic cancer vaccine in TRAMP mice with spontaneous prostate tumors, which subsequently translated to significant improvements in overall survival (Ardiani et al., supra).

In another embodiment, the androgen deprivation therapy is abiraterone, which is formulated as abiraterone acetate and marketed as ZYTIGA™ by Janssen Biotech, Inc. Abiraterone inhibits CYP17A1, a rate-limiting enzyme in androgen biosynthesis. Inhibition of CYP17A1 subsequently blocks the production of androgen in all endocrine organs, including the testes, adrenal glands, and in prostate tumors (Harris et al., Nature Clinical Practice Urology, 6(2): 76-85(2009)). In a phase III study in patients with CRPC previously treated with docetaxel, abiraterone was shown to improve overall survival by 3.9 months compared to placebo (de Bono et al., New England J. Med., 364(21): 1995-2005(2011)). Abiraterone is indicated for use in combination with prednisone to treat CRPC.

The term “immunotherapy,” as used herein refers to the treatment of a disease by inducing, enhancing, or suppressing an immune response. Immunotherapies designed to elicit or enhance an immune response are referred to as activation immunotherapies, while immunotherapies designed to suppress an immune response are referred to suppression immunotherapies. Types of immunotherapies include, but are not limited to, immunomodulators, cell-based immunotherapies, monoclonal antibodies, radiopharmaceuticals, and vaccines. Immunotherapy strategies for cancer are described in, for example, Waldmann, T. A., Nature Medicine, 9: 269-277 (2003)

Immunomodulators can be recombinant, synthetic, or natural substances that include, but are not limited to, cytokines (e.g., TNF-α, IL-6, GM-CSF, IL-2, and interferons), co-stimulatory molecules (e.g., B7-1 and B7-2), chemokines (e.g., CCL3, CCL26, CXCL7), glucans, and oligodeoxynucleotides.

Cell-based immunotherapies typically involve removal of immune cells (e.g., cytotoxic T-cells, natural killer cells, or antigen presenting cells (APCs)) from a subject, modification (e.g., activation) of immune cells, and return of the modified immune cells to the patient. In the context of the inventive method, the cell-based immunotherapy desirably is Sipuleucel-T (PROVENGE™), which is an autologous active cellular immunotherapy used in the treatment of asymptomatic or minimally symptomatic CRPC (Plosker, G. L., Drugs, 71(1): 101-108 (2011); and Kantoff et al., New Engl. J. Med., 363: 411-422 (2010)).

Several monoclonal antibodies have been approved for the treatment of cancer, including naked antibodies and antibody-drug conjugates based on human, humanized, or chimeric antibodies (Scott et al., Nat Rev Cancer, 12(4): 278-87 (2012); Harding et al., MAbs, 2(3): 256-65 (2010); and Weiner et al., Nature Rev. Immunol., 10(5): 317-327 (2010)). In one embodiment, the inventive method comprises treating the prostate cancer cells with any suitable monoclonal antibody known in the art. Such monoclonal antibodies include, for example, ipilumimab (YERVOY™), which is a fully human antibody that binds to CTLA-4 and is indicated for the treatment of melanoma. Antibodies that target the interaction of programmed death receptor-1 (PD-1) with its ligands PD-L1 and PD-L2, also can be used in the invention (see, e.g., Weber, Semin. Oncol., 37(5): 430-4309 (2010); and Tang et al., Current Oncology Reports, 15(2): 98-104 (2013)). Antibodies that inhibit PD-1 signaling include, for example nivolumab (also known as BMS-936558 or MDX1106; see, e.g., ClinicalTrials.gov Identifier NCT00730639), and MK-3575 (see, e.g., Patnaik et al., 2012 American Society of Clinical Oncology (ASCO) Annual Meeting, Abstract # 2512). Monoclonal antibodies that specifically target prostate cancer are under development and also can be used in the invention (see, e.g., Jakobovits, A., Handb. Exp. Pharmacol., 181: 237-56 (2008); and Ross et al., Cancer Metastasis Rev., 24(4): 521-37 (2005)).

Radiopharmaceuticals are radioactive drugs which are currently used to treat and diagnose a variety of diseases, including cancer. For example, radionuclides can be targeted to antibodies (i.e., radioimmunotherapy) to treat blood-derived cancers (Sharkey, R. M. and Goldenberg, D. M., Immunotherapy, 3(3): 349-70 (2011)). Several radioisotopes have been approved to treat cancer, including iodine-125, iodine-131, and radium-223 (marketed as XOFIGO™). Radium-223 has been approved as a radiopharmaceutical to treat metastatic bone cancer and CRPC. In CRPC, radium-223 also has been shown to enhance the anti-tumor immune response.

Vaccines represent another strategy to prevent and treat cancer. Many different cancer vaccine platforms are currently being evaluated in phase II and/or phase III clinical trials, including, for example, peptide-based vaccines, recombinant viral vectors, killed tumor cells, or protein-activated dendritic cells (see, e.g., Schlom, J., J. Natl. Cancer. Inst., 104: 599-613 (2012)). Any suitable vaccine can be used in the inventive method. In one embodiment, the vaccine can be the PSA/TRICOM vaccine (PROSTVAC™), which is a cancer vaccine composed of a series of poxviral vectors engineered to express PSA and a triad of human T-cell costimulatory molecules (see, e.g., Madan et al., Expert Opin. Investigational Drugs, 18(7): 1001-1011 (2009); and U.S. Pat. Nos. 4,547,773; 6,045,802; 6,165,4,60; 6,548,068; 6,946,133; 7,247,615; 7,368,116; 7,598,225; 7,662,395; 7,871,986; and 8,178,508). In another embodiment, the vaccine can be a Brachyury vaccine, which comprises recombinant yeast or poxvirus that has been genetically modified to express the Brachyury transcription factor (see, e.g., International Patent Application Publications WO 2014/043518 and WO 2014/043535; and U.S. Pat. Nos. 8,188,214 and 8,613,933).

The invention also provides a method of reducing breast cancer cell growth, which method comprises treating breast cancer cells with a combination of endocrine deprivation therapy and immunotherapy, whereupon growth of the breast cancer cells is reduced. The term “breast cancer” is synonymous with the term “breast carcinoma,” and refers to cancer that forms in tissues of the breast or mammary gland. “Breast cancer cells” refer to cells obtained or derived from a breast cancer. In another embodiment, the inventive method can be used to inhibit growth of hyperplastic, but not malignant, breast cells, such as, for example, usual hyperplasia or atypical hyperplasia.

The breast cancer cells also can be of any grade or stage, as determined by a variety of factors including tumor size, lymph node status, estrogen-receptor and progesterone-receptor levels in the tumor tissue, human epidermal growth factor receptor 2 (HER2/neu) status, menopausal status, and the general health of the patient. Cancer staging and grading guidelines are described in detail in, e.g., Edge et al. (eds.), American Joint Committee on Cancer (AJCC) Staging Manual, 7^{th} Edition (2010), or the SEER Program Coding and Staging Manual, NIH Publication Number13-5581, U.S. Department of Health and Human Services National Cancer Institute (2013).

In one embodiment, the breast cancer cells can have been subjected to one or more breast cancer therapies (e.g., surgery, chemotherapy, and/or radiation) prior to the inventive method. In another embodiment, the breast cancer cells have become resistant to other standard treatment regimens. For example, the breast cancer cells can be resistant to chemotherapy and/or radiation therapy.

The breast cancer cells can be positive or negative for an androgen receptor (AR). As discussed above, AR is widely expressed in breast cancers and has been proposed as a therapeutic target in estrogen-receptor (ER) negative breast cancers that express AR (Cochrane et al., Breast Cancer Res., 16: R7 (2014)). In one embodiment, the breast cancer cells express an androgen receptor. Alternatively, the breast cancer cells do not express an androgen receptor. The breast cancer cells also can be positive or negative for an estrogen receptor (ER). The estrogen receptor is a ligand-activated transcription factor composed of several domains that are important for hormone binding, DNA binding, and activation of transcription. The ER is activated by 17β-estradiol, and binding of estrogen to the ER stimulates proliferation of mammary cells. The estrogen receptor is overexpressed in about 70% of breast cancers (referred to as “ER-positive” breast cancers). In one embodiment, the breast cancer cells express an estrogen receptor. Alternatively, the breast cancer cells do not express an estrogen receptor.

The term “endocrine deprivation therapy” (also referred to as “hormonal therapy”), as used herein, refers to a treatment for breast cancer in which the level of endocrine hormones, such as estrogen and/or testosterone, in a patient are reduced, typically by pharmaceutical or surgical methods (see, e.g., Angelopoulos et al., Endocr. Relat. Cancer, 11: 523-535 (2004); Dhingra, K., Invest. New Drugs, 17(3): 285-311 (1999); and Garay, J. P. and Park, B. H., Am. J. Cancer Res., 2(4): 434-445 (2012)). Surgical approaches to endocrine deprivation include oophorectomy. Pharmaceutical approaches to endocrine deprivation therapy include estrogen inhibitors and androgen inhibitors. In one embodiment, the endocrine deprivation therapy is an androgen inhibitor such as, for example, cyproterone acetate, abiraterone, abiraterone acetate (ZYTIGA™), or enzalutamide (XTANDI™). The androgen inhibitor preferably is abiraterone or enzalutamide. Alternatively or additionally, the endocrine deprivation therapy is an estrogen inhibitor, such as, for example, megestrol (MEGACE™), an aromatase inhibitor (e.g., anastrozole), a selective estrogen receptor down-regulator (SERD) (e.g., fulvestrant), a gonadotropin-releasing hormone (GnRH) analogue, or a selective estrogen receptor modulator (SERM) (e.g., tamoxifen or raloxifene). The estrogen inhibitor preferably is tamoxifen.

Tamoxifen is a selective estrogen receptor modulator (SERM) which is indicated for the treatment of metastatic breast cancer in women and men and ductal carcinoma in situ. Tamoxifen a nonsteroidal agent that binds to estrogen receptors (ER), inducing a conformational change in the receptor, which results in a blockage or change in the expression of estrogen-dependent genes. Prolonged binding of tamoxifen to the nuclear chromatin of estrogen-dependent genes results in reduced DNA polymerase activity, impaired thymidine utilization, blockade of estradiol uptake, and decreased estrogen response. Like most SERMs, tamoxifen is antiestrogenic in breast tissue, but is estrogenic in the uterus and bone. Tamoxifen is described in detail in, for example, Jordan, V. C., Br J Pharmacol., 147 (Suppl 1): S269-76 (2006); and U.S. Pat. No. 4,536,516.

In the inventive method of reducing growth of breast cancer cells, the breast cancer cells can be treated with any suitable immunotherapy, such as those described herein. Desirably, the breast cancer cells are treated with a monoclonal antibody or a vaccine. Any suitable monoclonal antibody for treatment of breast cancer can be used in the invention. Such monoclonal antibodies include, for example, trastuzumab (HERCEPTIN™), pertuzumab (PERJETA™), and the antibody-drug conjugate ado-trastuzumab emtansine (KADCYLA™) Preferably, the monoclonal antibody is trastuzumab (HERCEPTIN™). Any suitable vaccine for the treatment of breast cancer can be used in the invention, and several vaccines that specifically target breast cancer are currently under investigation (see, for example, Mittendorf et al., Ann Oncol. doi: 10.1093/annonc/mdu211 (2014); Huang et al., Proc. Natl. Acad. Sci. USA, 110(7): 2517-2522 (2013); and Toh et al., Cancer Res., 73(24 Suppl): Abstract nr P5-01-05 (2013)). The vaccine can be, for example, PANVAC, which is a cancer vaccine based on two viral vectors (recombinant vaccinia and recombinant fowlpox) expressing transgenes for the tumor-associated antigens epithelial mucin 1 and carcinoembryonic antigen (see, e.g., Madan et al., Expert Opin Biol Ther., 7(4): 543-54; International Patent Application Publications WO 2005/046622 and WO 2005/046614; and U.S. Pat. Nos. 5,698,530; 6,001,349; 6,319,496; 6,969,609; 7,211,432; 7,368,116; 7,410,644; 7,771,715; 7,999,071; and 8,609,395). In another embodiment, the vaccine can be a Brachyury vaccine, which comprises a recombinant yeast or poxvirus that has been genetically modified to express the Brachyury transcription factor (see, e.g., International Patent Application Publications WO 2014/043518 and WO 2014/043535; and U.S. Pat. Nos. 8,188,214 and 8,613,933). The vaccine also can be a yeast MUC-1 immunotherapeutic, such as those described in, e.g., U.S. Patent Application Publication 2013/0315941 and International Patent Application Publication WO 2012/103658.

The combination of immunotherapy and androgen or endocrine deprivation therapy reduces or inhibits growth of prostate cancer cells or breast cancer cells, respectively. The term “growth,” as used herein, encompasses any aspect of the growth, proliferation, and progression of prostate or breast cancer cells, including, for example, cell division (i.e., mitosis), cell growth (e.g. increase in cell size), an increase in genetic material (e.g., prior to cell division), and metastasis. Reduction, inhibition, or suppression of cancer cell growth includes, but is not limited to, inhibition of cancer cell growth as compared to the growth of untreated or mock treated cells, inhibition of proliferation, inhibition of metastases, sensitization to immune-mediated killing (e.g., T-cell-mediated lysis), induction of cancer cell senescence, induction of cancer cell death, and reduction of tumor size.

The prostate cancer cells and the breast cancer cells can be in vivo or in vitro. The term “in vivo” refers to a method that is conducted within living organisms in their normal, intact state, while an “in vitro” method is conducted using components of an organism that have been isolated from its usual biological context (e.g., isolating and culturing cells obtained from an organism). Preferably, the prostate cancer cells and breast cancer cells are in vivo, and exist within a human male prostate cancer patient and a human male or female breast cancer patient, respectively. When the prostate cancer cells or the breast cancer cells are in vivo, i.e., in a human, the inventive methods induce a therapeutic effect in the prostate cancer subject or breast cancer subject and treat the prostate cancer or the breast cancer. As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method comprises administering a “therapeutically effective amount” of the immunotherapy and the androgen deprivation therapy (e.g., enzalutamide or abiraterone) or endocrine deprivation therapy (e.g., tamoxifen). A “therapeutically effective amount” refers to an amount effective, at dosages and for periods of time necessary, to achieve a desired therapeutic result. The therapeutically effective amount may vary according to factors such as the disease state, age, and weight of the individual, and the ability of the immunotherapy and androgen or endocrine deprivation therapy to elicit a desired response in the individual.

The following examples further illustrate the invention but, of course, should not be construed as in any way limiting its scope.

### EXAMPLE 1

This example demonstrates that treatment of prostate cancer cells with androgen deprivation therapy increases their sensitivity to T-cell lysis.

Enzalutamide has previously been shown to induce immunogenic modulation in TRAMP-C2 mouse prostate carcinomas and to improve tumor cells' sensitivity to gp70-specific CTL killing in vitro (Ardiani et al., Clin. Cancer Res., 19(22): 6205-6218 (2013)) The effects of ADT with enzalutamide or abiraterone on human prostate carcinomas were investigated. To determine the effect of ADT on tumor-cell proliferation, two human prostate tumor-cell lines; LNCaP (AR+, HLA-A2) and PC-3 (AR−, HLA-A24), were treated in vitro with vehicle (DMSO) or 10 μM enzalutamide or abiraterone This clinically relevant dose was similar to or lower than the median plasma concentration achieved in humans (Richards et al., Cancer Res., 72(9): 2176-2182 (2012)). Cells were harvested 24, 48, or 72 h after exposure, and the total number of adherent viable cells was determined by trypan blue exclusion. Viability was confirmed by 7AAD staining.

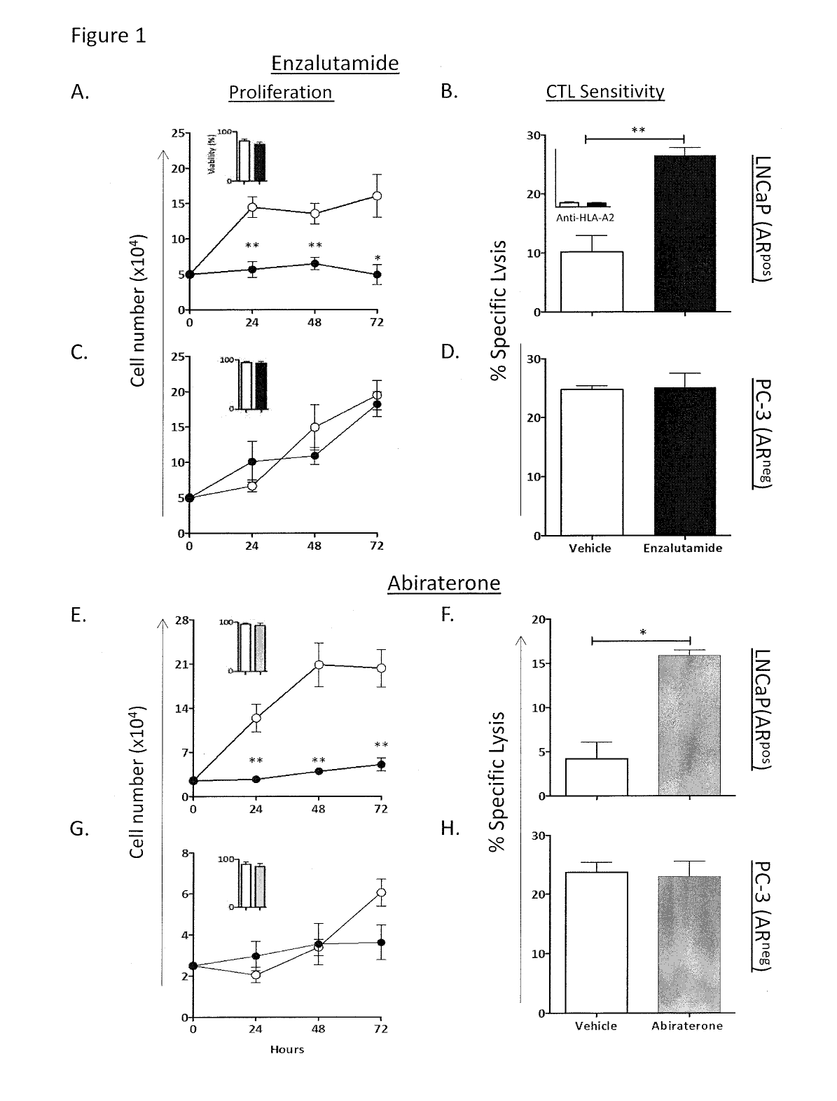

Treatment with enzalutamide significantly inhibited the growth of LNCaP cells (P<0.01) (FIG. 1A), but did not inhibit the proliferation of PC-3 cells (FIG. 1C). Similarly, abiraterone significantly reduced the proliferation of LNCaP cells (P<0.01), but did not affect PC-3 cells (FIGS. 1E and 1G). Neither enzalutamide nor abiraterone affected the viability of LNCaP and PC-3 cells, as measured by trypan blue exclusion after 3 days of drug exposure (insets, FIGS. 1A, 1C, 1E, and 1G). To determine whether enzalutamide or abiraterone mediated increased sensitivity to T-cell lysis, LNCaP and PC-3 cells were treated with either drug in vitro and used as target cells for MUC1-specific CTL-mediated killing assays. Cytotoxicity assays were performed as previously described (Ardiani et al., supra). In particular, tumor cells were treated with vehicle or 10 μM enzalutamide or abiraterone. At specific time points, cells were harvested and counted. Equal numbers of effector target cells from all treatment were plated with respective cytotoxic T-cells. The E:T ratio was held at 30:1 and adherent cells were used as targets in a standard cytotoxicity assay using indium-111 (GE Health Care, Vienna, Va.).

Exposing LNCaP cells to enzalutamide significantly enhanced their sensitivity to MUC1-specific CTL-mediated lysis relative to tumor cells exposed to vehicle (P<0.01) (FIG. 1B). This killing was MHC-restricted as determined by HLA-A2 blocking (FIG. 1B inset). Similarly, exposing LNCaP cells to abiraterone significantly improved their sensitivity to MUC1 -specific CTL-mediated lysis compared to vehicle-treated tumor cells (P<0.05) (FIG. 1F). However, neither enzalutamide nor abiraterone improved PC-3 cells' sensitivity to MUC1-specific CTL-mediated lysis (FIGS. 1D and 1H) relative to vehicle-treated tumor cells.

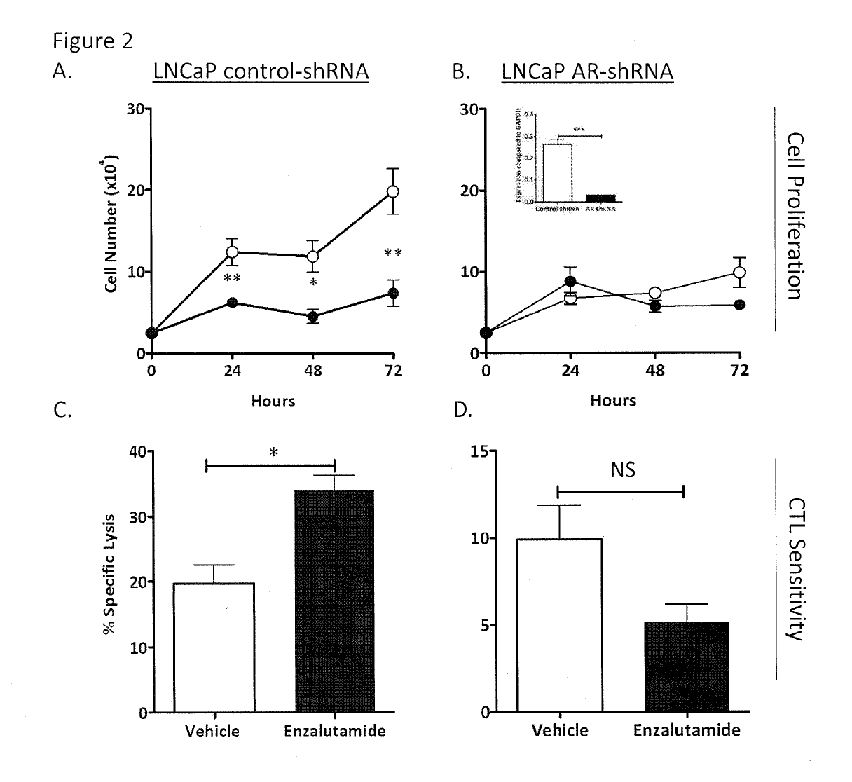

To confirm that immunogenic modulation by enzalutamide is AR-dependent, a pair of LNCaP cell lines stably expressing either control-shRNA (expresses AR) or AR-shRNA cells (reduced or no AR expression) (Cheng et al., Cancer Res., 66(21): 10613-10620 (2006)) were tested. In vitro, enzalutamide significantly inhibited the proliferation of LNCaP control-shRNA (P<0.01) (FIG. 2A) but did not affect the growth of LNCaP AR-shRNA (FIG. 2B). Treatment with enzalutamide significantly enhanced the sensitivity of LNCaP control-shRNA to CEA-specific CTL-mediated lysis compared to vehicle-treated tumor cells (P<0.05) (FIG. 1C). The improved sensitivity to T-cell killing mediated by enzalutamide was lost when tumors with reduced expression of AR were used, as seen in FIG. 2D, where LNCaP AR-shRNA treated with enzalutamide or vehicle demonstrated similar sensitivity to CEA-specific CTL-mediated lysis. RT-PCR confirmed the reduced expression of AR in LNCaP cells stably expressing AR-shRNA compared to LNCaP expressing control-shRNA (inset, FIG. 2B).

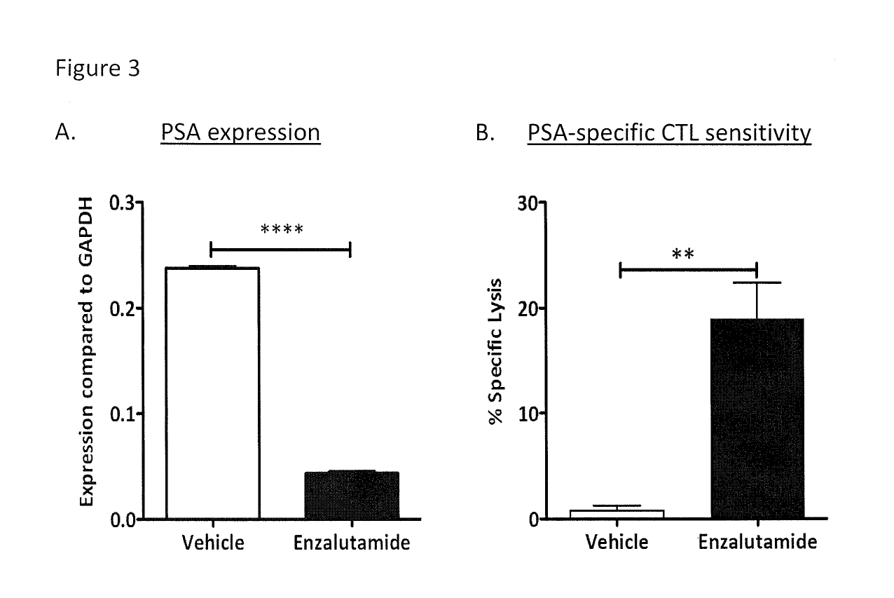

Enzalutamide has been shown to reduce PSA levels both in vitro and in the clinic (Scher et al., New England J. Med., 367(13): 1187-1197 (2012); and Chen et al., The Lancet Oncology, 10(10): 981-91 (2009)), thus it was investigated whether reduced levels of PSA would inhibit tumor sensitivity to a PSA-specific immune response in patients undergoing immunotherapy. To determine the effect of enzalutamide on PSA levels, LNCaP cells were treated in vitro with enzalutamide for 48 hours. RT-PCR analysis showed a 5.5-fold reduction in levels of PSA mRNA (P<0.0001) (FIG. 3A). When these cells were used as targets for PSA-specific CD8+ T-cell killing (FIG. 3B), treatment with enzalutamide significantly improved their sensitivity to PSA-specific T-cell killing, despite the reduction in PSA level (i.e., reduction in CTL targets) (P<0.01).

The results of this example demonstrate that both enzalutamide and abiraterone mediate immunogenic modulation in human prostate tumor cells in an AR-dependent manner, and that enzalutamide reduced PSA levels while improving sensitivity to PSA-specific CTL killing.

### EXAMPLE 2

This example demonstrates that androgen deprivation therapy modulates expression of apoptosis genes in vitro and in vivo.

The properties of ADT that induce immunogenic modulation and sensitize prostate tumor cells to immune-mediated attack are novel and have not been previously described. It was hypothesized that modulation of apoptotic genes might be part of the mechanism of action of ADT-induced CTL sensitization. The expression of 96 genes involved in the process of apoptosis was analyzed by RT-PCR in LNCaP cells treated with enzalutamide or abiraterone in vitro. In particular, gene expression was assessed using an apoptosis PCR array (SA Biosciences, Valencia, Calif.) as per the manufacturer's instructions. RT-PCR was performed on the 7300 Real-Time PCR System (Applied Biosystems, Carlsbad, Calif.). Where indicated, values were calculated as expression relative to GAPDH, as previously described (Ardiani et al., Cancer Res., 74(7): 1945-1957 (2014))

Of these 96 genes, 3 were up-regulated and 12 were down-regulated greater than 2-fold by enzalutamide treatment. Abiraterone treatment resulted in a greater than 2-fold up-regulation of 11 genes and down-regulation of 14 genes. Further analysis showed that only 9 genes were down-regulated by both enzalutamide and abiraterone (Table 1).

TABLE 1

| Gene | Name | Function | Enzalutamide | Abiraterone |
|---|---|---|---|---|
| CASP1 | Caspase 1 | Proapoptosis | −2.17 | −4.05 |
| CD27 | CD27 molecule | Proapoptosis | −2.26 | −3.50 |
| HRK | Harakiri, BCL-2 | Pr-apoptosis | −2.86 | −8.57 |
| | interacting | | | |
| | protein | | | |
| CASP8 | Caspase 8 | Proapoptosis | −2.96 | −2.25 |
| CASP5 | Caspase 5 | Proapoptosis | −3.50 | −4.22 |
| TNFSF8 | Tumor necrosis | Proapoptosis | −3.50 | −7.51 |
| | factor (ligand) | | | |
| | super family, | | | |
| | member 8 | | | |
| TNFRSF25 | Tumor necrosis | Proapoptosis | −3.70 | −7.41 |
| (DR3) | factor receptor | | | |
| | super family, | | | |
| | member 25 | | | |
| DAPK1 | Death-associated | Proapoptosis | −6.53 | −13.73 |
| | protein kinase 1 | | | |
| NAIP | NLR family, | Antiapoptosis | −13.90 | −4.72 |
| | apoptosis | | | |
| | inhibitory protein | | | |

Among these 9 genes, one in particular, NAIP, was down-regulated 14-fold by enzalutamide and 5-fold by abiraterone treatment. To examine the reduced expression of NAIP in vivo, either LNCaP or PC-3 cells were transplanted into female nude mice (Charles River, Wilmington, Mass.) (s.c. in the right flank with 5×10⁶ LNCaP or PC-3 prostate tumor cells). Once tumors reached 500 mm³, mice were left untreated or treated with 10 mg/day of enzalutamide. After 7 days of treatment, tumors were subjected to immunohistochemistry staining to detect the presence and intensity of NAIP expression. Specifically, NAIP expression was detected via immunohistochemistry using a rabbit polyclonal antibody to NAIP (Novus Bio, Littleton, Colo.) according to the manufacturer's instructions. Entire slides were digitally scanned by an Aperio ScanScope CS system and analyzed by Aperio ImageScope Viewer software (Aperio Technologies Inc., Vista, Calif.). Statistical analysis was performed using 3-7 murine tumors, each prepared as a complete stained tumor section. Positive tumor regions were determined using the Positive Pixel Count v9 algorithm. Negative controls included omission of primary antibody with PBS and matched rabbit isotype antibody. In all cases, necrotic areas of tumor were excluded from analysis.

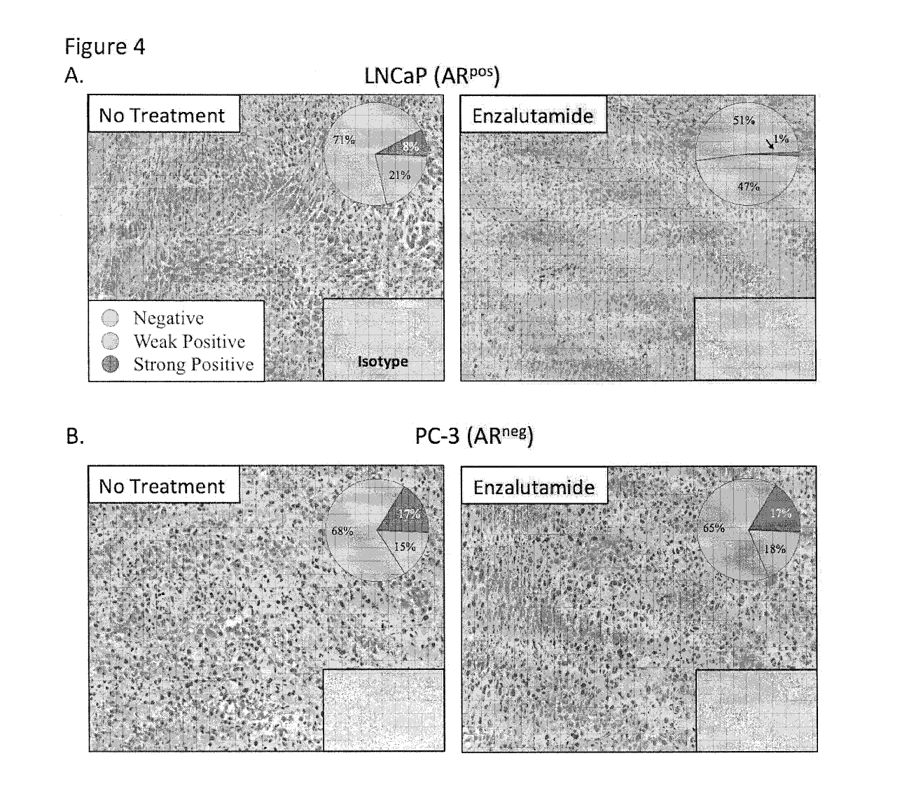

NAIP showed much less intense staining in LNCaP tumors harvested from mice treated with enzalutamide (FIG. 4A, right panel) compared to LNCaP tumors harvested from untreated mice (FIG. 4A, left panel). Positive pixel analysis (insets, FIG. 4A) from 2 independent experiments demonstrated a significant 2- to 8-fold reduction in tumor cells that strongly stained for NAIP in enzalutamide-treated tumors compared to untreated tumors (P<0.01). Furthermore, the overall population of enzalutamide-treated tumor cells expressing NAIP significantly decreased by 1.5-fold (P<0.01) compared to untreated tumors, and there was a significant 2.2-fold increase (P<0.01) in tumor area that did not express NAIP in enzalutamide-treated tumors compared to untreated tumors. In contrast, in mice harboring PC-3 cells treatment with enzalutamide did not mediate significant changes in NAIP expression. Similarly intense staining was seen in harvested tumors from enzalutamide-treated and untreated PC-3 tumors (FIG. 4B). Positive pixel analysis demonstrated similar NAIP expression in enzalutamide-treated and untreated PC-3 tumors (insets, FIG. 4B).

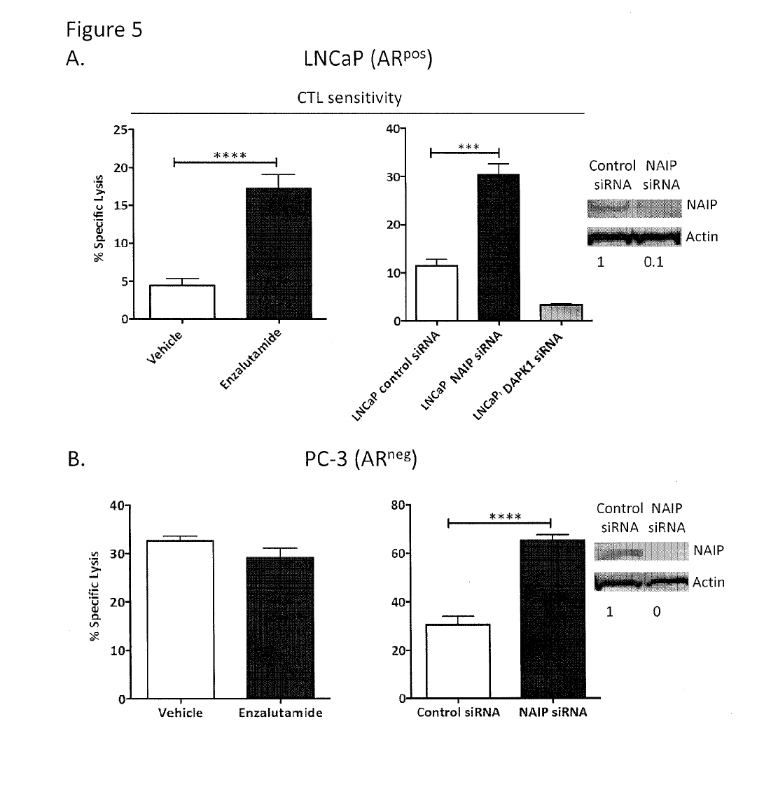

To investigate the role of NAIP in immunogenic modulation and subsequent improvement in CTL sensitivity mediated by enzalutamide, the expression of NAIP was transiently reduced in LNCaP cells in vitro using NAIP siRNA, and this reduced expression was confirmed by western blot (inset, FIG. 5A). In particular, siRNA duplexes targeting NAIP sequences and control were purchased from Origene (Rockville, Md.). LNCaP or PC-3 cells were transfected with NAIP siRNA or control siRNA according to the manufacturer's instructions. The interference of NAIP expression was confirmed by RT-PCR using TaqMan probes for NAIP (Hs03037952_m1, Applied Biosystems) or western blot.

NAIP expression in LNCaP cells transfected with NAIP siRNA was reduced by 90% 48 hours post-transfection, compared to LNCaP cells transfected with control siRNA. In a parallel experiment, LNCaP cells were treated with vehicle or enzalutamide for 48 hours and used as targets for CEA-specific CTL lysis. As shown in FIGS. 1A and 5A (left panel), treatment with enzalutamide, previously shown to reduce NAIP expression in vitro (Table 1) and in vivo (FIG. 4A), significantly enhanced the sensitivity of LNCaP cells to CEA-specific CD8+ T-cell killing (P<0.0001). Similarly, reduced expression of NAIP in LNCaP cells treated with NAIP siRNA also significantly increased tumor cells' sensitivity to T-cell-mediated killing (P<0.001) (FIG. 5A, right panel). These data suggest that NAIP played a major role in immunogenic modulation. Besides NAIP, another gene of interest was death-associated protein kinase 1 (DAPK1), as enzalutamide down-regulated this gene 6.5-fold (Table 1). To evaluate the importance of DAPK1, DAPK1 siRNA was used to reduce the expression of DAPK1. Reduced expression of DAPK1 did not increase sensitivity of LNCaP cells to T-cell killing. This suggested that DAPK1 did not play a major role in enzalutamide-mediated immunogenic modulation (FIG. 5A, right panel).

To validate the importance of NAIP in the process of immunogenic modulation, PC-3 cells previously shown to be unaffected by enzalutamide (FIG. 1B) were transfected with either control or NAIP siRNA for 48 hours, and reduced expression of NAIP was confirmed by western blot (inset, FIG. 5B). PC-3 cells were also independently treated with vehicle or enzalutamide. Forty-eight hours after enzalutamide or siRNA treatment, the cells were used as targets for MUC1-specific T-cell killing. As previously shown, enzalutamide did not improve the sensitivity of PC-3 cells to T-cell killing (FIG. 5B, left panel). However, reduced expression of NAIP in PC-3 cells mediated a significant improvement in T-cell-mediated killing (P<0.0001) (FIG. 5B, right panel).

The results of this example demonstrate that the NAIP gene is involved in the molecular mechanism of enzalutamide-mediated immunogenic modulation.

### EXAMPLE 3

This example demonstrates that enzalutamide improves the sensitivity of LNCaP/AR(cs) cells to T-cell-mediated killing.

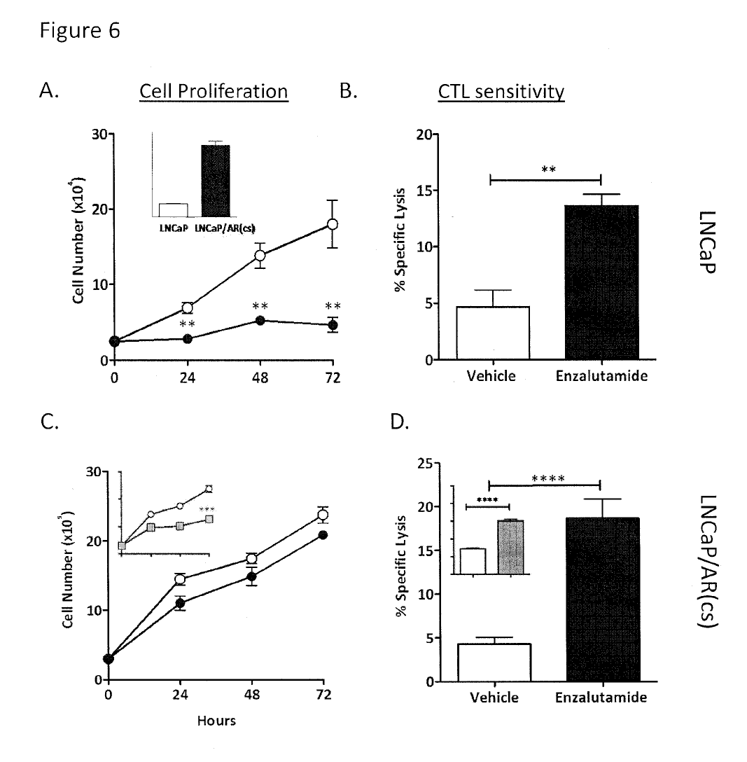

Because CRPC is commonly associated with increased expression of AR, arising from amplification or mutation of the AR gene as well as other mechanisms (Niu et al., Proc. Natl. Acad. Sci. USA, 105(34): 12182-12187 (2008); and Visakorpi et al., Nature Genetics, 9(4): 401-406 (1995)), it was investigated whether enzalutamide-mediated immunogenic modulation could improve the sensitivity of prostate tumor cells engineered to overexpress AR to T-cell-mediated killing. RT-PCR confirmed the overexpression of AR by 5-fold in (LNCaP/AR(cs)) cells (inset, FIG. 6A). To determine the effect of enzalutamide on cell proliferation, LNCaP and LNCaP/AR(cs) cells were treated in vitro with vehicle (DMSO) or 10 μM enzalutamide (FIG. 6B). Treatment with 10 μM enzalutamide significantly inhibited the growth of LNCaP cells (P<0.01) (FIG. 6A), but did not inhibit the proliferation of LNCaP/AR(cs) cells (FIG. 6C). A higher, but still clinically feasible, dose of enzalutamide (30 μM) inhibited the growth of LNCaP/AR(cs) cells by 50% while having no effect on the cells' viability (FIG. 6C, inset). To determine whether enzalutamide could increase the sensitivity of these ADT resistant cells to T-cell killing, LNCaP/AR(cs) cells were treated with 10 μM enzalutamide in vitro and used as target cells in either CEA- or PSA-specific CD8+ T-cell-mediated killing assays. Exposing LNCaP (FIG. 6B) and LNCaP/AR(cs) (FIG. 6D) cells to 10 μM enzalutamide significantly improved the cells' sensitivity to T-cell killing (P<0.01). Exposure of LNCaP/AR(cs) cells to (30 μM) also significantly improved the cells' sensitivity to T-cell killing (P<0.01). There was no significant difference in the improved CTL sensitivity by treatment of the of LNCaP/AR(cs) cells with 10 μM or 30 μM enzalutamide.

The results of this example demonstrate that enzalutamide enhances sensitivity to immune-mediated killing of prostate tumor cells that overexpress AR.

### EXAMPLE 4

This example provides a description of materials and methods used in Examples 5-10. Examples 5-10 demonstrate that both enzalutamide and abiraterone inhibit breast cancer cell tumor growth and enhance sensitivity of breast cancer cells to T-cell mediated killing. The increased sensitivity to immune-mediated killing occurs irrespective of the tumor expression of the intended target, AR.

Tumor Cells

ZR75-1, BT549 and MDA MB 231 breast cancer cells were purchased from American Type Culture Collection (Manassas, Va.) in 2014 and were maintained at low passage number (<5). All cells were maintained in RPMI-1640 medium supplemented with 10% fetal bovine serum, and 1% of HEPES, penicillin/streptomycin, L-glutamine, non-essential amino acids and sodium pyruvate. In addition, BT-549 cells required 10 μg/ml human insulin. All cells were regularly tested for Mycoplasma contamination and were discarded after 12 passages.

Drug Preparation

For in vitro studies, enzalutamide and abiraterone (Selleck Chemicals, Houston, Tex.) were dissolved in DMSO (vehicle, Sigma Aldrich, St. Louis, Mo.) to a concentration of 10 mM and stored at −20° C. A dose of 10 μM of either enzalutamide or abiraterone was used for all in vitro experiments where media and drug or vehicle were replaced daily.

RNA Isolation, Quantitative Real-Time PCR and Apoptosis Array

Quantitative real-time PCR was used to evaluate the AR expression levels of untreated ZR75-1, BT549 and MDA MB 231 breast cancer cells. Total RNA was isolated from the cells using the RNeasy Extraction Kit (Qiagen, Valencia, Calif.). RNA was reverse-transcribed into cDNA using the Advantage RT-for-PCR Kit (Clontech, Mountain View, Calif.). cDNA (10 ng) was used in a quantitative real-time (RT) PCR reaction using probes specific for AR (Hs00901571_m1) and GAPDH (4326317E). AR mRNA expression level was calculated as expression relative to GAPDH. To evaluate the effect of enzalutamide on apoptosis-associated gene expression, MDA-MB-231 cells were treated with either enzalutamide or vehicle for 24 hours. Total RNA was isolated from the cells using the RNeasy Extraction Kit. RNA was reverse-transcribed into cDNA using the RT² First Strand Kit (SA Biosciences, Valencia, Calif.). Relative mRNA expression levels of 90 genes involved in apoptosis were assessed using an apoptosis PCR array (SA Biosciences) per the manufacturer's instructions. RT-PCR was performed on the 7300 Real-Time PCR System (Applied Biosystems, Carlsbad, Calif.).

Western Blotting

AR expression was confirmed by western blot using a rabbit monoclonal antibody to AR (Abeam, Cambridge, Mass.) and a mouse monoclonal antibody to β-actin (Cell Signaling, Danvers, Mass.). Untreated ZR75-1, BT549 and MDA MB 231 cells were lysed using Cell Lysis Buffer containing 1 mM PMSF (Cell Signalling, Danvers, Mass.) and 10 μL/mL HALT′ Protease/Phosphatase Inhibitor Cocktail (Thermo Scientific, Rockford, Ill.) according to the manufacturer's protocol. Protein concentration was measured using a BCA Protein Assay Kit (Thermo Scientific). Aliquots containing 50 μg of protein were run on a Bolt 4%-12% gradient Bis-Tris gel using the Bolt system then transferred to a PVDF membrane using the iBLOT 2 Transfer System (Life Technologies, Grand Island, N.Y.). Membranes were blocked overnight at 4° C. with PBS containing 5% BSA and 0.05% Tween20, then incubated with primary antibodies in block for 4 hours at room temperature. Membranes were then incubated with IRDye-labeled goat anti-rabbit and goat anti-mouse secondary antibodies (LI-COR Biosciences, Lincoln, Nebr.) at a 1:10000 dilution in block for 1 hour at room temperature. Membranes were imaged using the Odyssey Infrared Imaging System (LI-COR Biosciences).

Tumor Cell Proliferation

To evaluate the effect of enzalutamide on breast cancer cell proliferation, ZR75-1, BT549 and MDA MB 231 cells were treated with either enzalutamide or vehicle (DMSO) for 24, 48, or 72 hours. At the indicated time points, cells were harvested and the number of viable cells was determined by trypan blue exclusion.

Flow Cytometry

To assess the effect of enzalutamide on cell surface phenotype of breast cancer cell, ZR75-1, BT549 and MDA MB 231 cells were treated with either enzalutamide or vehicle for 48 hours. After 48 hours, cells were harvested and stained with the following antibodies: HLA A2-PE-Cy7, MUC-1-FITC, CD54-BV421, CD95-FITC (BD Biosciences, San Jose, Calif.), CEA-APC (Miltenyi Biotec, Auburn, Calif.), TRAIL receptor 1 and TRAIL receptor 2 (R & D Systems, Minneapolis, Minn.). LIVE/DEAD Fixable Violet Dead Cell Stain (Life Technologies, Grand Island, N.Y.) was used to determine cell viability. Cells were incubated with the antibodies for 30 min at 4° C., acquired on a FACS Verse flow cytometer (Becton Dickinson, Franklin Lakes, N.J.), and analyzed using FlowJo software (TreeStar, Inc., Ashland, Oreg.).

Cytotoxic T Lymphocyte & TRAIL Killing Assays

To determine the ability of androgen deprivation therapy to alter the sensitivity of ZR75-1, BT549 and MDA MB 231 cells to CTL or TRAIL-mediated lysis, cells were treated with enzalutamide, abiratirone, or vehicle for 48-72 hours, after which they were harvested and used as targets in standard lysis assays. Cells were labeled with ¹¹¹In-labeled oxyquinoline (Medi-Physics Inc., Arlington Heights, Ill.) and coincubated in 96-well round-bottom plates at 37° C./5% CO2 with HLA-A2-restricted carcinoembryonic antigen (CEA)-specific CTLs at an effector:target ratio of 30:1 or 500 ng/ml KillerTRAIL (Enzo Life Sciences, Farmingdale, N.Y.). The HLA-A2-restricted CEA-specific CTL recognizes the CEA peptide epitope YLSGANLNL (SEQ ID NO: 1) (CAP-1). After 18 hours, supernatants were harvested and analyzed for the presence of ¹¹¹In using a WIZARD2 Automatic Gamma Counter (PerkinElmer, Waltham, Mass.). The percentage of tumor lysis was calculated as follows: % tumor lysis=[(experimental cpm−spontaneous cpm)/(maximum cpm−spontaneous cpm)]×100.

ELISA

The level of secreted OPG was confirmed in MDA MB 231 cells treated with 10 μM enzalutamide or vehicle for 48 hours using a DuoSet ELISA (R & D Systems) according to the manufacturer's instructions. ELISA was performed on combined supernatant samples taken following 24 and 48 hours of treatment.

RNA Interference (siRNA)

OPG expression was inhibited in MDA MB 231 cells using siRNA duplexes targeting OPG sequences and control siRNA duplexes (Origene, Rockville, Md.). MDA-MB-231 cells were transfected with OPG or control siRNA according to the manufacturer's instructions. The interference of OPG expression was confirmed 48 hours post siRNA transfection by ELISA as described. Forty-eight hours post siRNA transfection, the MDA MB 231 cells were also used as target cells in CEA-specific CTL and TRAIL-mediated lysis assays as described.

### EXAMPLE 5

This example demonstrates that androgen deprivation therapy reduces the proliferation of AR positive breast cancer cells.

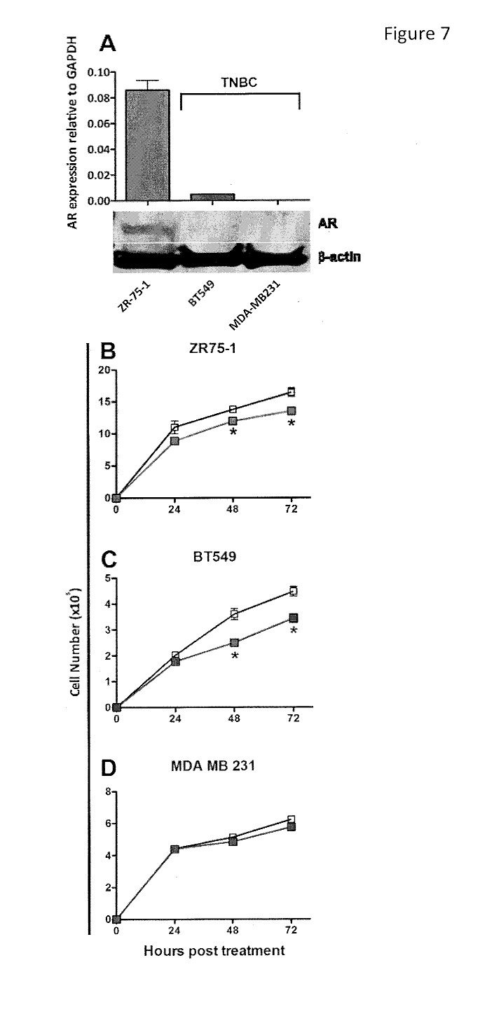

The effects of enzalutamide on breast carcinoma cells that represent three major classifications of breast cancer (luminal B (ZR75-1), mesenchymal-like (BT549) and mesenchymal stem-like (MDA MB 231)) were determined. These cell lines also represent different combinations of estrogen receptor and androgen receptor positivity. ZR75-1 cells (ER+) displayed a high degree of AR expression as determined by qRT-PCR and Western Blot; BT549 cells (ER−) expressed AR but at a much lower degree; and MDA MB 231 (ER−) cells expressed no AR (FIG. 7A).

To determine the effect of enzalutamide on the proliferation of the breast cancer cell lines, each cell line was exposed to vehicle (DMSO) or 10 μM enzalutamide for 24, 48 or 72 hours. This dose of enzalutamide mimics the clinically achievable median plasma concentration and was the dose shown to induce immunogenic modulation in prostate cancer cells (see Ardiani et al., Oncotarget, 5(19): 9335-9348 (2014); and Richards et al., Cancer Research, 72(9): 2176-2182 (2012)).

After the designated period of treatment, cells were harvested and counted, and their viability was measured by trypan blue exclusion. Enzalutamide significantly inhibited the proliferation of ZR75-1 (ER+AR+) cells (P<0.05, FIG. 7B) and to a greater degree that of BT549 (ER−AR+) cells (P<0.01, FIG. 7C) after 48 or 72 hours of treatment compared to vehicle control.

### EXAMPLE 6

This example demonstrates that enzalutamide modulates the expression of tumor cell markers associated with immune recognition.

It has been shown previously that radiation and chemotherapy can alter the cell surface phenotype of human tumor cells, rendering them more sensitive to T cell-mediated killing (see Gameiro et al., Cancer Biotherapy & Radiopharmaceuticals, 27(1): 23-25 (2012); and Gameiro et al., Oncotarget, 5(2): 403-416 (2014)). To determine if enzalutamide could modify the expression of cell-surface markers that influence immune recognition, breast carcinoma cells were treated with vehicle or 10 μM enzalutamide for 48 hours, then stained and analyzed them by flow cytometry. The results are set forth in Table 1.

TABLE 1
Modulation of breast tumor phenotype by enzalutamide

| | HLA A2 | CEA | MUC-1 | ICAM-1 | Fas | Trail R1 | Trail R2 |
|---|---|---|---|---|---|---|---|
| ZR75-1 | | | | | | | |
| Vehicle | 19.0 (2591) | 24.3 (4936) | 58.2 (2492) | 32 (1965) | 33.2 (1070) | 13.8 (5374) | 7.6 (2972) |
| Enzalutamide | 10.0 (3047) | 28.9 (4690) | 37.1 (2203) | 26.1 (2473) | 26.8 (1611) | 7.9 (4325) | 1.4 (4502) |
| BT549 | | | | | | | |
| Vehicle | 86.6 (2450) | 12.4 (5685) | 30.6 (996) | 32.0 (4709) | 26.8 (568) | 14.6 (598) | 38.1 (1011) |
| Enzalutamide | 87.6 (2532) | 15.3 (3443) | 25.5 (930) | 26.1 (6239) | 15.8 (566) | 23.6 (562) | 49.5 (1110) |
| MDA MB 231 | | | | | | | |
| Vehicle | 97.8 (31622) | 0.02 (773) | 14.2 (589) | 93.4 (5486) | 8.8 (331) | 13.8 (769) | 68.3 (1130) |
| Enzalutamide | 96.4 (21175) | 9.7 (626) | 11.1 (503) | 95.3 (7254) | 9.0 (375) | 19.2 (1125) | 70.5 (1516) |

Flow cytometric analysis of surface marker expression on breast cancer cell lines exposed to enzalutamide. ZR75-1, BT549 and MDA MB 231 cells were exposed to 10 μM enzalutamide for 48 hours then analyzed by flow cytometry for HLA A2, CEA, MUC-1, ICAM-1, Fas, and TRAIL receptors 1 and 2. Percent positivity and mean fluorescence intensity, in parentheses, are shown.
Values in bold denote an increase of >20% relative to vehicle-treated cells.

Treatment with enzalutamide significantly increased the expression of MHC-I, ICAM-1, Fas and Trail receptor 2 on ZR75-1 (ER+AR+) cells (see Table 1). Enzalutamide treatment also upregulated the expression of ICAM-1 as well as Trail receptors 1 and 2 and the tumor associated antigen CEA on BT549 (ER−AR+) cells (see Table 1). Surprisingly, however, enzalutamide similarly upregulated CEA, ICAM-1 and Trail receptors 1/2 on MDA MB 231 (ER−AR−) cells (see Table 1). Previous studies have suggested that improving the expression of any one of these markers could be capable of making tumor cells more amenable to T cell-mediated killing (see Gameiro et al., Cancer Biotherapy & Radiopharmaceuticals, 27(1): 23-25 (2012); Gameiro et al., Oncotarget, 5(2): 403-416 (2014); Chakraborty et al., J. Immunol., 170(12): 6338-6347 (2003); and Chakraborty et al., Cancer Research, 64(12): 4328-4337 (2004)).

### EXAMPLE 7

This example demonstrates that enazlutamide increases the sensitivity of breast cancer cells to immune-mediated lysis regardless of AR expression.

To determine whether enzalutamide was capable of increasing the sensitivity of breast cancer cells to T-cell lysis, ZR75-1 (ER+AR+), BT549 (ER−AR+) and MDA MB 231 (ER−AR−) cells were treated with vehicle or 10 μM enzalutamide and used as target cells for CTL-mediated killing assays utilizing CEA-specific CTLs.

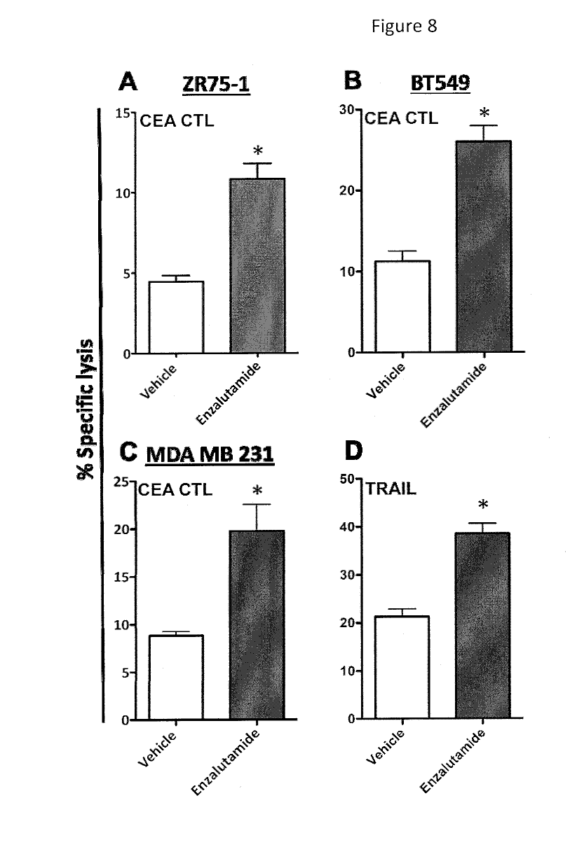

Exposing ZR75-1 (ER+AR+) cells to enzalutamide significantly enhanced their sensitivity to CEA-specific CTL-mediated lysis compared to the vehicle control (P<0.01, FIG. 8A). Similarly, exposing BT549 (ER−AR+) cells to enzalutamide significantly improved their sensitivity to CEA-specific CTL-mediated lysis relative to vehicle-treated cells (P<0.01, FIG. 8B). However, enzalutamide also was capable of increasing the sensitivity of MDA MB 231 (ER−AR−) cells to CEA-specific CTL-mediated lysis compared to vehicle-treated tumor cells (P<0.05, FIG. 8C). Cytotoxic T cells can cause target cell lysis by multiple mechanisms including the release of perform and granzyme, the binding of Fas ligand on the T cell to Fas on the target cell, and the binding of Trail on the T cell to Trail receptors on the target cell all resulting in the induction of the apoptosis cascade. Both triple negative breast cancer (TNBC; ER−PR−Her2−) cell lines, BT549 (AR+) and MBA MB 231 (AR−), displayed an upregulation of Trail receptors in the absence of Fas upregulation (see Table 1).

To confirm the effect of enzalutamide on MDA MB 231 (ER−AR−) cells and to further investigate the effect of enzalutamide-induced Trail receptor upregulation, the cells were treated with vehicle or 10 μM enzalutamide and analyzed for their sensitivity to Trail-mediated lysis. Again, enzalutamide significantly improved the sensitivity of MDA MB 231 (ER−AR−) cells to Trail-mediated lysis (P<0.01, FIG. 8D).

These results indicate that enzalutamide-mediated immunogenic modulation in human breast carcinoma cells leads to their improved sensitivity to immune-mediated killing, and that this effect is independent of AR expression. This improvement also occurs in triple negative breast cancer cells (TBNC), which are ER−, PR−, and Her2− (e.g., BT549 and MBA MB 231 cells).

### EXAMPLE 8

This example demonstrates that abiraterone also improves the sensitivity of breast cancer cells to CTL-mediated lysis regardless of AR expression.

To evaluate whether a second form of ADT also was capable of increasing the sensitivity of breast cancer cells to T-cell lysis, 10 μM abiraterone or vehicle (DMSO) was used to treat ZR75-1 (ER+AR+) and MDA MB 231 (ER−AR−) cells. These cells were then used as target cells for CTL-mediated killing assays utilizing CEA-specific CTLs.

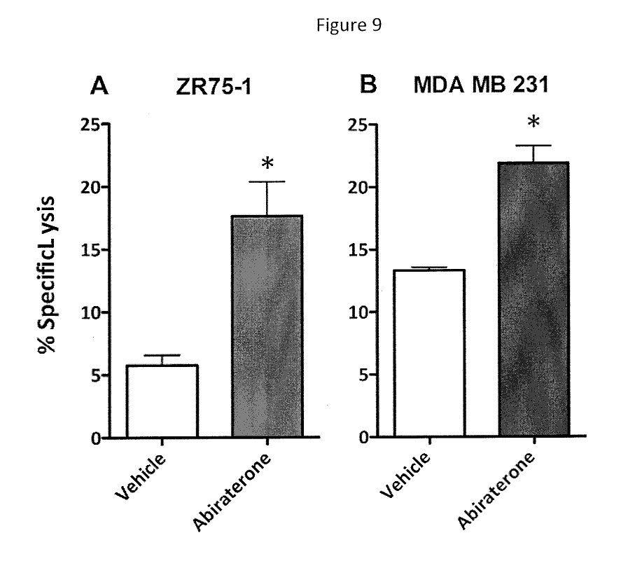

As with enzalutamide, this dose of abiraterone mimics the clinically achievable median plasma concentration and was the dose shown to induce immunogenic modulation in prostate cancer cells (see Ardiani et al., Oncotarget, 5(19): 9335-9348 (2014); and Richards et al., Cancer Research, 72(9): 2176-2182 (2012)). Following treatment with abiraterone, ZR75-1 (ER+AR+) cells (P<0.05, FIG. 9A) and MDA MB 231 (ER−AR−) cells (P>0.01, FIG. 9B) both displayed enhanced sensitivity to CTL-mediated lysis compared to vehicle-treated cells.

These results indicate that multiple types of ADT could successfully mediate improved immune-mediated lysis of human breast tumor cells regardless of their AR expression.

### EXAMPLE 9

This example demonstrates that enzalutamide significantly reduces the expression of osteoprotegerin in AR-TNBC MDA MB 231 cells.

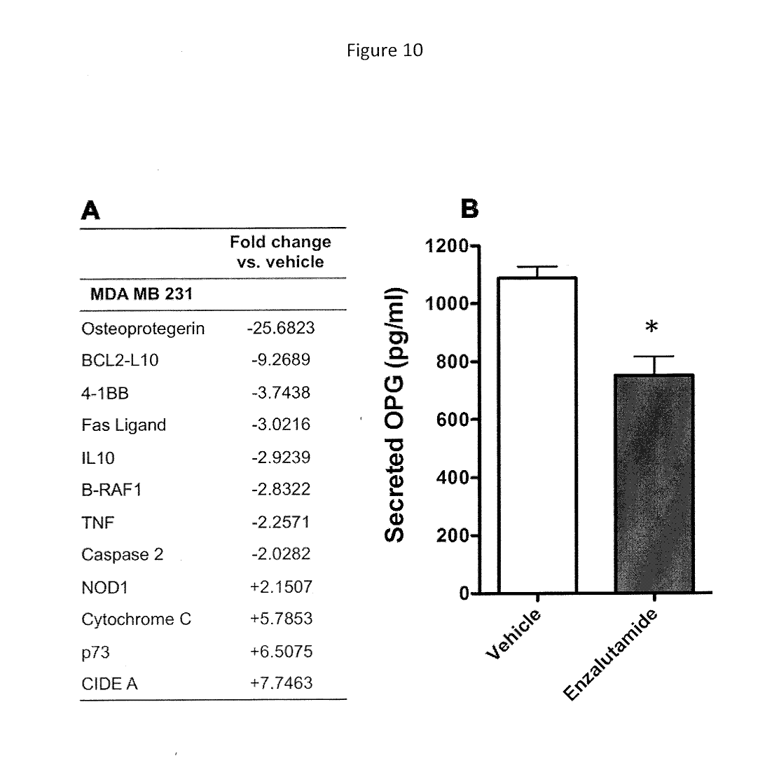

Enzalutamide has been shown to alter pro and anti-apoptotic gene expression in prostate cancer cells (see Ardiani et al., Oncotarget, 5(19): 9335-9348 (2014)). To examine whether enzalutamide has a similar effect in breast cancer cells, which could play a role in its ability to increase their sensitivity to immune-mediated lysis, the expression of 90 genes involved in the apoptotic process was examined by qRT-PCR in enzalutamide treated MDA MB 231(ER−AR−) cells. Of these genes, 4 were up-regulated and 8 were down-regulated >2-fold by enzalutamide treatment relative to the expression observed in vehicle-treated cells. Among these genes, osteoprotegerin (OPG), an antiapoptotic gene, was down-regulated ˜25-fold by enzalutamide (FIG. 10A). OPG is a decoy receptor for receptor activator of nuclear factor kappa-B ligand (RANKL) which inhibits RANKL activation of nuclear factor kappa-B induced apoptosis.

To verify that this reduction in OPG mRNA resulted in reduced expression of OPG protein, an ELISA for secreted OPG confirmed that 10 μM enzalutamide indeed reduced the amount of OPG expressed by MDA MB 231(ER−AR−) cells (P<0.05, FIG. 10B) was performed.

### EXAMPLE 10

This example demonstrates that modulation of OPG recapitulates the improvement in sensitivity to immune-mediated lysis observed in enzalutamide treated ER−AR− MDA MB 231 cells.

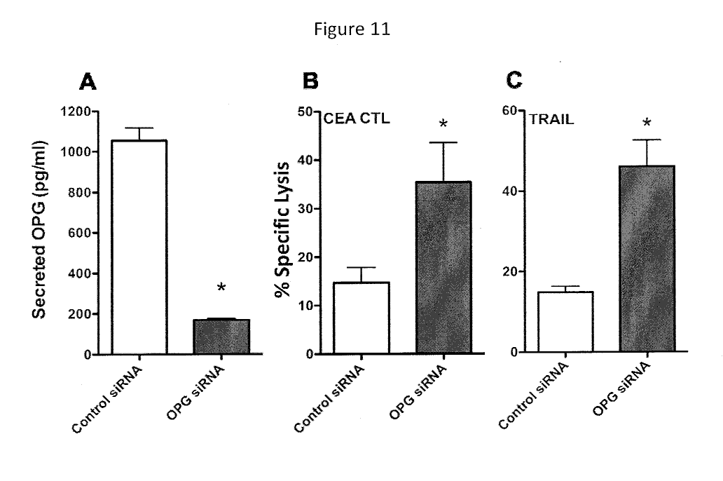

To determine the role of OPG in the increase in CTL sensitivity mediated by enzalutamide, the expression of OPG in MDA MB 231(ER−AR−) cells was transiently reduced using siRNA. An ˜80% reduction in secreted OPG was confirmed by ELISA 48 hours post-OPG siRNA transfection relative to control siRNA transfected cells (FIG. 11A).

These MDA MB 231(ER−AR−) cells were then evaluated for their sensitivity to CTL and Trail-mediated killing. Similar to the results achieved with enzalutamide treatment, a reduction in OPG expression led to improved sensitivity of MDA MB 231(ER−AR−) cells to both CEA-specific CTL-mediated lysis (P<0.05, FIG. 11B) and Trail-mediated lysis (P<0.01, FIG. 11C).

These results indicate that the reduction in OPG expression played a major role in the increased sensitivity to immune-mediated killing which resulted from enzalutamide treatment.

### EXAMPLE 11

This example demonstrates that tamoxifen treatment of breast cancer cells increases sensitivity to immune-mediated lysis regardless of their estrogen receptor (ER) status.

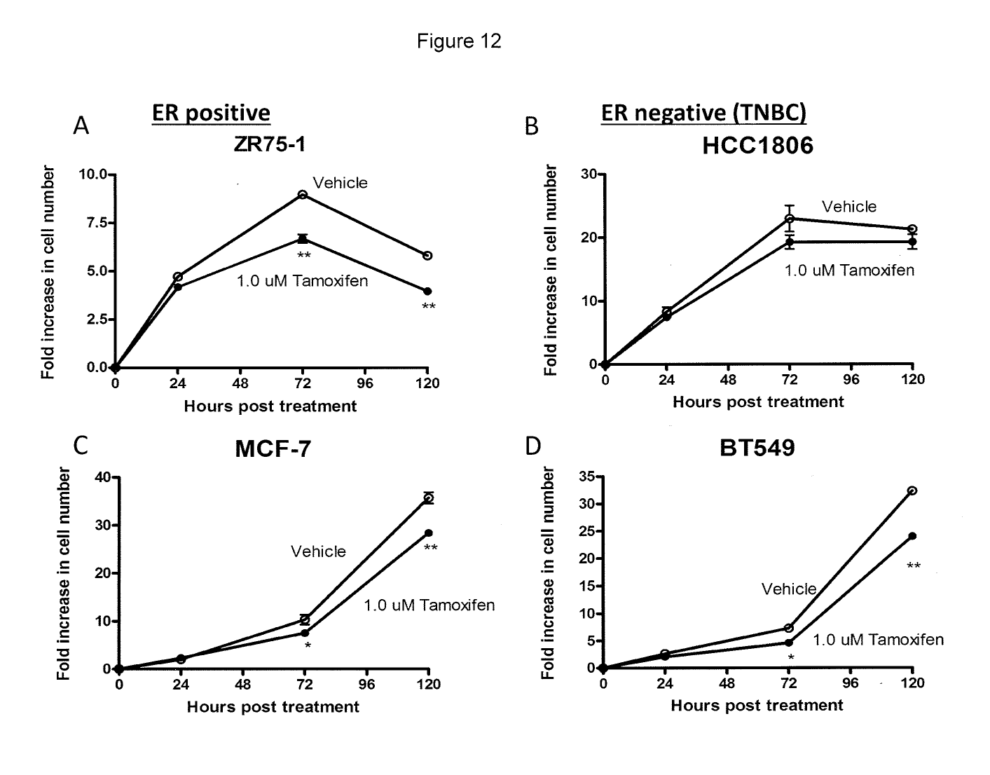

ZR75-1 (ER+PR+Her2+), MCF (ER+PR+Her2−), BT549 (ER−PR−Her2−), and HCC1806 (ER−PR−Her2−) cells were treated with vehicle or 1 μM tamoxifen to replicate intratumoral concentrations. FIGS. 12A-12D illustrate the fold increase in cell number following treatment. Viability never diminished more than 10% at any given time point.

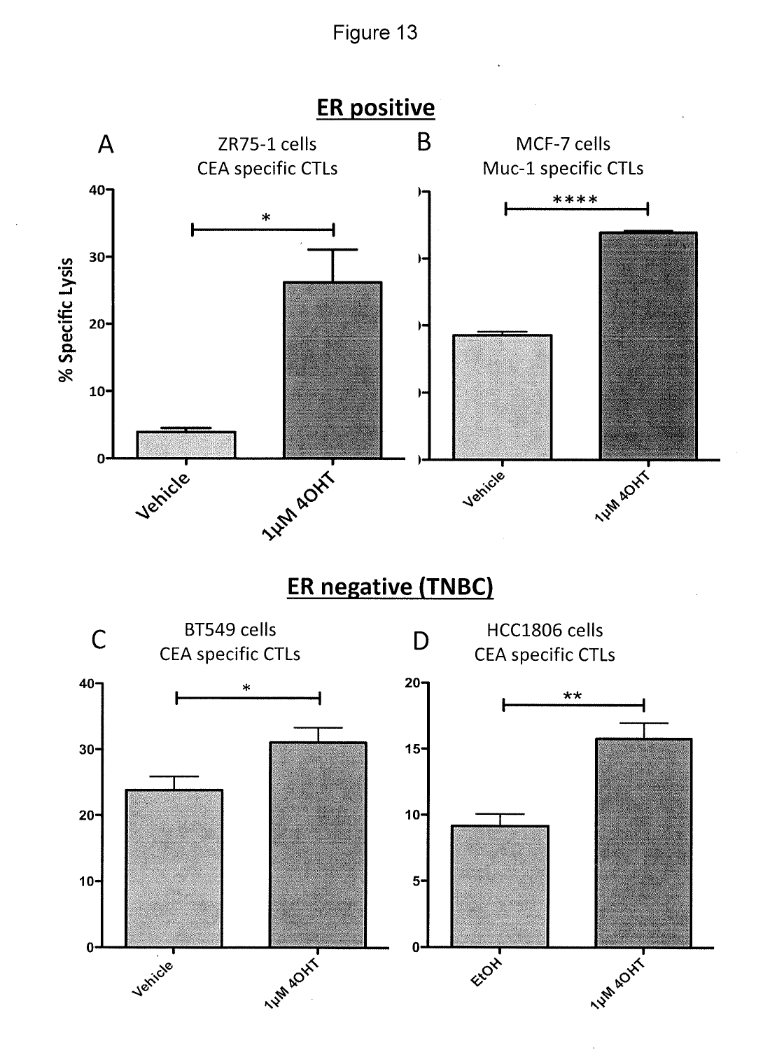

To determine sensitivity to immune-mediated killing, breast cancer cells treated with vehicle or 1 μM tamoxifen for 72 hours and then used as targets in a CTL assay using CEA-specific or MUC-1 specific CTLs as effector cells at an E:T ratio of 30:1. As illustrated in FIGS. 13A-13D, tamoxifen improved the CTL-mediated lysis of ER+ cell lines (MCF-7 and ZR75-1), and improved CEA-specific killing of ER− cell lines (BT549 and HCC1806).

These results demonstrate that tamoxifen treatment influences the CTL sensitivity of breast cancer cells independent of the tumor expression of the intended target of tamoxifen, ER. This improvement also occurs in triple negative breast cancer cells (TBNC), which are ER−, PR−, and Her2− (e.g., BT549 and HCC1806 cells).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of reducing prostate cancer cell growth, which method comprises treating prostate cancer cells with a combination of androgen deprivation therapy and immunotherapy, whereupon growth of the prostate cancer cells is reduced.

2. The method of claim 1, wherein the prostate cancer cells are metastatic castration resistant prostate cancer (CRPC) cells.

3. The method of claim 1, wherein the prostate cancer cells are resistant to chemotherapy and/or radiation therapy.

4. The method of claim 1, wherein the prostate cancer cells are resistant to treatment with androgen deprivation therapy.

5. The method of claim 1, wherein the androgen deprivation therapy is an androgen inhibitor.

6. The method of claim 5, wherein the androgen inhibitor is enzalutamide.

7. The method of claim 5, wherein the androgen inhibitor is abiraterone.

8. The method of claim 1, wherein the immunotherapy is a vaccine.

9. The method of claim 8, wherein the vaccine is the PSA/TRICOM vaccine (PROSTVAC™).

10. The method of claim 1, wherein the prostate cancer cells are in vivo.

11. The method of claim 10, wherein the prostate cancer cells are in a human.

12. The method of claim 1, wherein the prostate cancer cells are in vitro.

13. The method of claim 1, wherein the prostate cancer cells express an androgen receptor.

14. A method of reducing prostate cancer cell growth, which method comprises treating prostate cancer cells with a combination of abiraterone and immunotherapy, whereupon growth of the prostate cancer cells is reduced.

15. The method of claim 14, wherein the prostate cancer cells are metastatic castration resistant prostate cancer (CRPC) cells.

16. The method of claim 14, wherein the prostate cancer cells are resistant to chemotherapy and/or radiation therapy.

17. The method of claim 14, wherein the prostate cancer cells are resistant to treatment with androgen deprivation therapy.

18. The method of claim 14, wherein the immunotherapy is a vaccine, a monoclonal antibody, a cell-based immunotherapy, or a radiopharmaceutical.

19. The method of claim 18, wherein the immunotherapy is the PSA/TRICOM vaccine (PROSTVAC™), a Brachyury vaccine, Sipuleucel-T (PROVENGE™), ipilumimab, nivolumab, or radium-223 (XOFIGO™)

20. The method of claim 14, wherein the prostate cancer cells are in vivo.

21. The method of claim 20, wherein the prostate cancer cells are in a human.

22. The method of claim 14, wherein the prostate cancer cells are in vitro.

23. The method of claim 14, wherein the prostate cancer cells express an androgen receptor.

24. A method of reducing breast cancer cell growth, which method comprises treating breast cancer cells with a combination of endocrine deprivation therapy and immunotherapy, whereupon growth of the breast cancer cells is reduced.

25. The method of claim 24, wherein the breast cancer cells are resistant to chemotherapy and/or radiation therapy.

26. The method of claim 24, wherein the endocrine deprivation therapy is an androgen inhibitor.

27. The method of claim 26, wherein the androgen inhibitor is enzalutamide.

28. The method of claim 26, wherein the androgen inhibitor is abiraterone.

29. The method of claim 24, wherein the endocrine deprivation therapy is an estrogen inhibitor.

30. The method of claim 29, wherein the estrogen inhibitor is tamoxifen or an aromatase inhibitor.

31. The method of claim 24, wherein the immunotherapy is a vaccine or a monoclonal antibody.

32. The method of claim 31, wherein the immunotherapy is PANVAC, a yeast-MUC-1 immunotherapeutic, a Brachyury vaccine, and trastuzumab (HERCEPTIN™).

33. The method of claim 24, wherein the breast cancer cells are in vivo.

34. The method of claim 33, wherein the breast cancer cells are in a human.

35. The method of claim 24, wherein the breast cancer cells are in vitro.

36. The method of claim 24, wherein the breast cancer cells express an androgen receptor.

37. The method of claim 24, wherein the breast cancer cells do not express an androgen receptor.

38. The method of claim 24, wherein the breast cancer cells express an estrogen receptor.

39. The method of claim 24, wherein the breast cancer cells do not express an estrogen receptor.
